(19) United States
(12) Patent Application Publication
(10) Pub. No.: US 2026/0301097 A1
(43) Pub. Date: Oct. 1, 2026

(54) NETWORK OPTIMIZATION BASED ON QUANTUM POSITIONING OF USER DEVICES

(71) Applicants: AT&T Intellectual Property I, L.P., Atlanta, GA (US); AT&T Mobility II LLC, Atlanta, GA (US)

(72) Inventors: Mark Stockert, San Antonio, TX (US); Jerry Robinson, Middletown, NJ (US); Vinay Sharma, Levittown, NY (US); Thomas J. Routt, Sequim, WA (US); Yuan Ding, Plainsboro, NJ (US)

(21) Appl. No.: 19/091,686

(22) Filed: Mar. 26, 2025

(51) Int. Cl. G06Q 50/26; G06F 18/26; G06N 10/60; G06Q 10/04; G16H 40/67

(52) U.S. Cl. CPC G06Q 50/26; G06F 18/26; G06N 10/60; G06Q 10/043; G16H 40/67

(57) ABSTRACT

A method performed by a processing system including at least one processor includes collecting, from a quantum sensor in a communications network, position data for a public safety device, tracking a movement pattern and a usage pattern of the public safety device by providing the position data as input to a quantum machine learning model that generates the movement pattern and the usage pattern as an output, determining, based on an analysis of the movement pattern and the usage pattern, that an emergency service should be contacted, and placing a call to the emergency service.

The present disclosure relates generally to cellular networks, and relates more particularly to devices, non-transitory computer-readable media, and methods for optimizing fifth generation/sixth generation and next-generation networks based on quantum positioning of user devices.

### BACKGROUND

Network optimization is the process of improving network performance, reliability, efficiency, and/or scalability. Network optimization may include procedures for performing resource allocation, load balancing, interference management, and other operations.

### SUMMARY

The present disclosure broadly discloses methods, computer-readable media, and systems for optimizing fifth generation/sixth generation and next-generation networks based on quantum positioning of user devices. In one example, a method performed by a processing system including at least one processor includes collecting, from a quantum sensor in a communications network, position data for a public safety device, tracking a movement pattern and a usage pattern of the public safety device by providing the position data as input to a quantum machine learning model that generates the movement pattern and the usage pattern as an output, determining, based on an analysis of the movement pattern and the usage pattern, that an emergency service should be contacted, and placing a call to the emergency service.

In another example, a non-transitory computer-readable medium may store instructions which, when executed by a processing system including at least one processor, cause the processing system to perform operations. The operations may include collecting, from a quantum sensor in a communications network, position data for a public safety device, tracking a movement pattern and a usage pattern of the public safety device by providing the position data as input to a quantum machine learning model that generates the movement pattern and the usage pattern as an output, determining, based on an analysis of the movement pattern and the usage pattern, that an emergency service should be contacted, and placing a call to the emergency service.

In another example, a device may include a processing system including at least one processor and a non-transitory computer-readable medium storing instructions which, when executed by the processing system, cause the processing system to perform operations. The operations may include collecting, from a quantum sensor in a communications network, position data for a public safety device, tracking a movement pattern and a usage pattern of the public safety device by providing the position data as input to a quantum machine learning model that generates the movement pattern and the usage pattern as an output, determining, based on an analysis of the movement pattern and the usage pattern, that an emergency service should be contacted, and placing a call to the emergency service.

### BRIEF DESCRIPTION OF THE DRAWINGS

The teachings of the present disclosure can be readily understood by considering the following detailed description in conjunction with the accompanying drawings, in which:

FIG. 1A illustrates an example system in which examples of the present disclosure for optimizing fifth generation/sixth generation and next-generation networks based on quantum positioning of user devices may operate;

FIG. 1B illustrates a block diagram showing the network optimizer of FIG. 1A in greater detail, including connections of the network optimizer to other elements of the system of FIG. 1A;

FIG. 2 illustrates a flowchart of an example method for optimizing the provision of emergency services in a communications network, in accordance with the present disclosure; and

FIG. 3 illustrates an example of a computing device, or computing system, specifically programmed to perform the steps, functions, blocks, and/or operations described herein.

To facilitate understanding, similar reference numerals have been used, where possible, to designate elements that are common to the figures.

### DETAILED DESCRIPTION

The present disclosure broadly discloses methods, computer-readable media, and systems for optimizing fifth generation/sixth generation and next-generation networks based on quantum positioning of user devices. As discussed above, network optimization is the process of improving network performance, reliability, efficiency, and/or scalability. Network optimization may include procedures for performing resource allocation, load balancing, interference management, and other operations. Effective network optimization improves the customer experience while minimizing waste of network resources.

With the advent of Fifth Generation (5G) and developing Sixth Generation (6G)/next-generation networks, there is a growing need for efficient network management to handle the increasing number of connected devices and the demand for high-speed, low latency communications. Conventional network optimization methods, however, rely on approximate location data, which can result in suboptimal resource allocation, load balancing, and interference management.

Examples of the present disclosure utilize quantum positioning to obtain precise and real-time location data of users of communications networks (e.g., 5G, 6G, and next-generation communication networks). The location data may be used in combination with quantum computing, quantum machine learning, and agent-based systems that leverage foundation models to enhance the optimization of the communications network for resource allocation, load balancing, and interference management, resulting in increased network efficiency, improved network performance, and greater network scalability. Among other applications, examples of the present disclosure can enhance public safety applications by ensuring reliable and efficient communications for emergency services and first responders.

6G mobile networks are projected to provide an increase of approximately 100 times in volumetric spectral and energy efficiency (measured in bits per second per Herz per cubic meter, or bps/Hz/m³) relative to 5G networks and to incorporate a large connectivity-based structure. Current specifications for 6G networks require, at minimum: (1) bandwidth that is high enough to enable high-fidelity holographic immersive communications, wireless data centers, multi-Terahertz (THz) frequencies, and multi-terabits per second (Tbps) uplink/downlink (UL/DL) speeds; (2) wide enough coverage to support large scale Internet of Things (IoT) networks with multi-gigabits per second (Gbps) to Tbps coverage; (3) enhanced reliability relative to 5G (e.g., in excess of 99.99% or 99.999% availability); (4) high endpoint density (incorporating, e.g., up to 10 million devices per square kilometer); (5) hyper-synchronization of multiple parallel flows to multiple devices supporting synchronized parallel media streams which originate from a plurality of network endpoints and midpoints; (6) lower power consumption for resource-constrained devices; (7) time-sensitive operations (e.g., bounded latency, jitter, etc.); (8) tactile Internet (e.g., an evolution of IoT incorporating human-to-machine and machine-to-machine real-time interactive communications for remote robotic surgery with haptic robots and other applications); (9) smartdust (i.e., a system of multiple micro-electromechanical systems (MEMS) such as sensors and robots that can detect vibration, light, temperature, magnetism, and chemicals); (10) quantum electromechanical systems (QEMS) (e.g., nano-fabricated mechanical systems that incorporate transducers operating at the quantum limit to detect the magnetic moment of a single spin, deformation forces on a single macromolecule, and other events related to information and biomolecular technologies); (11) holographic communications (e.g., fully immersive virtual user experiences in which a key component is the interactive transmission of three-dimensional holographic images between or among one or multiple sources to one or multiple endpoints); (12) ubiquitous services (e.g., services based on seamlessly integrated connectivity architecture which may include land-, sea-, air-, and/or space-based nodes and may include a ‘z’ axis added to a two-dimensional surface-based routing/switching infrastructure to generate real-time three-dimensional location-based situational awareness); (13) cyber-physical systems and manufacturing (e.g., integrating the digital and real worlds such that intelligent objects interconnect and interact, such as massive IoT or MIoT); (14) ubiquitous first responder/emergency services (e.g., directed to highly available, reliable, resilient, ubiquitous, and network-agnostic frameworks for providing data, video, voice, graphical, and/or image integration, on-demand temporal coverage, network redundancy, in-building solutions, location-centric on-demand capacity, support for mission-critical push-to-talk, mission-critical data, and mission-critical video via remotely controlled and autonomous assets including connected vehicles, drones, and robots within an emergency services architecture); and (15) precise location tracking (e.g., to enable precise location tracking of user devices, vehicles, tracking along six degrees of freedom for virtual reality/augmented reality/mixed reality, holographic communications tracking including the three dimensions of spatial movement along x-, y-, and z-axes plus pitch, yaw, and rotation, and highly integrated space-air-ground integrated networking for tracking of public safety first responder assets including personnel, vehicles, and user equipment).

To provide the above features, 6G networks are expected to require quantum and hybrid quantum-classical communications to facilitate connecting a plurality of end-to-end quantum and hybrid quantum-classical networked application resources (e.g., application programs, application programming interfaces, application servers, security servers, data repositories/lakes, routers, switches, load balancers, links, and the like).

Quantum data (e.g., data generated by quantum and hybrid quantum-classical computational runtime environments) are characterized by quantum superposition and quantum entanglement, and yields n-dimensional probability distributions that require exponential compute resources to process, represent, store, and connect. The presence of quantum and hybrid quantum-classical end-to-end networked application resources, in turn, results in an exponential increase in the requirements to incorporate quantum positioning systems (QPSs), quantum artificial intelligence (QAI), quantum machine learning (QML), quantum deep learning (QDL), quantum reinforcement learning (QRL), and quantum blockchains within 6G mobile and fixed communications networks.

Quantum computing and quantum networking have assumed strategic importance due to: (1) the fact that continuing cost-performance improvements in classical (i.e., non-quantum) processor memory, speed, and very large scale integrated (VLSI) substrate density packing are unsustainable (e.g., due to quantum effects that pervade the quantum scales at which electronic, photonic, and opto-electronic processors, devices, and network components are fabricated and process data; and (2) the worldwide increase in the volume (e.g., scale), variety (e.g., form, structured/unstructured, etc.), velocity (e.g., streaming), and veracity (e.g., uncertainty) of data (e.g., due to exponential improvements in computing, storage, cloud, and mobility network economies of scale and specialization).

Quantum computation stores information as quantum bits (or “qubits”), which are quantum generalizations of classical bits. Qubits can be represented as a two-to-n-level quantum system based on, for example, electronic/photonic spin and polarization, where: (1) the state of a qubit is a phase vector |ψ [symbol] (mathematical description of a quantum system, a complex-valued probability amplitude and the probabilities for possible results of measurements made on the system) in a linear superposition of states such as |ψ [symbol] =α|0 [symbol] +β|1 [symbol] ; (2) state vectors |0 [symbol] and |1 [symbol] are physical eigenstates of the logical observable and form a computational basis spanning a two-to-n dimensional Hilbert space (i.e., inner product space of two or more vectors, equal to the vector inner product between two or more matrix representations of those vectors containing |ψ [symbol] ; and (3) a collection of qubits comprises a multi-particle quantum system.

Quantum computation can pursue all computational trajectories simultaneously based on quantum superposition (i.e., integration of all states between 0 and 1), whereas classical computation proceeds in a serial fashion. Quantum logic gates form basic quantum circuits that operate on qubits, are reversible with a few exceptions (unlike classical logic gates), and are unitary operators, described as unitary matrices relative to basis states. Quantum algorithms utilize quantum circuit gates to manipulate states of quantum systems, just as classical algorithms utilize classical logic gates (represented as a sequence of Boolean gates) to perform classical (non-quantum) computational operations.

Emerging quantum networks are based on quantum communication channels that transmit qubits between physically distinct quantum or hybrid quantum-classical processors that are capable of performing quantum logic operations on qubits.

Quantum annealing is a quantum computing technique that is used to solve optimization problems by exploiting the principles of quantum mechanics. Unlike classical optimization methods (which often get trapped in local minima), quantum annealing leverages quantum superposition and tunneling to explore a vast solution space more effectively. This makes quantum annealing particularly well-suited for complex combinatorial optimization problems, such as problems encountered in network resource management, load balancing, and interference mitigation.

Quantum annealing involves mapping an optimization problem into a physical system of qubits. These qubits represent different possible states or solutions to the optimization problem. The physical system of qubits is initialized in a superposition of all possible states, representing all potential solutions simultaneously. The process then involves gradually evolving the physical system's Hamiltonian (i.e., a function that describes the total energy of the system) from an initial state to a final state that encodes the solution to the optimization problem. During this evolution, quantum tunneling allows the physical system to traverse energy barriers that would trap classical systems in local minima. As the physical system approaches the final state, the physical system settles into a configuration that represents the global minimum energy landscape, corresponding to the optimal solution to the problem.

Quantum annealing algorithms provide a powerful tool for solving the complex optimization problems inherent in network management. By leveraging quantum mechanics, these algorithms can efficiently explore large solution spaces and find optimal configuration for resource allocation, load balancing, and interference management.

Widespread deployment of wireless technology has enabled a large number of location-based services. Position determination and tracking of locations are important for these services in emergencies; however, accuracy, reliability, and scalability are challenges for existing position determination and location tracking techniques. During public safety incidents, first responders, public safety entities, and public safety agencies need to continually track and intercommunicate to ensure a coordinated incident response that results in improved outcomes.

The locations of first responders are a foundational public safety requirement, including, for example, while in vehicles, while on foot, while involved in search and rescue operations, while fighting wildfires, while restoring communications in the wake of a natural disaster, or while responding to an incident inside a high-rise building. Artificial intelligence (AI)-driven “big” data analytics can inform predictive, proactive optimization of public safety mobile networks and attendant location-based services resulting in improved situational awareness.

Location-based services and attendant situational awareness predominantly in use by public safety are based on the U.S. Department of Defense global positioning system (GPS) for navigation and time synchronization. GPS generally comprises a core constellation of thirty-one operational satellites in medium Earth orbit (MEO) at an altitude of approximately 20,200 kilometers (or 12,550 miles), arranged into six equally spaced orbital planes (where each plane contains four “slots” occupied by twenty-four baseline satellites, or a twenty-seven slot constellation) to ensure that at least four GPS satellites are viewable from any planetary locations.

User receipt of four GPS satellite signals and satellite space-time coordinates at time of transmission enable solving for unknown space-time coordinates based on trilateration, where received signals are continuous-wave circularly polarized radio frequency (RF) signals resident on two carrier frequencies in the L-band centered at approximately 1575.42 MHz and 1227.6 MHz, carrier frequency signals are modulated by a pseudorandom noise (PRN) code, and GPS receivers conduct phase-difference measurements (e.g., pseudorange measurements, or the phase difference between received PRN codes and identical copies of PRN codes replicated within GPS receivers). Highly accurate atomic clocks are used to synchronize telecommunications networks, GPS satellite navigation, and positioning systems. Positioning and navigation systems rely on GPS satellite and/or RF signals that may be blocked by buildings or other infrastructures, or can be jammed, spoofed, or denied, thereby preventing accurate navigation and positioning. GPS signals are only available outdoors. Indoor locations or areas with signal blockage require other methods to determine position. Fingerprinting based positioning methods rely on RF received signal strength (RSS) values from a variety of RF sources (e.g., Bluetooth, WiFi, 4G/5G, ZigBee, etc.) to estimate position. These methods rely on unpredictable radio propagation characteristics in indoor environments and areas with limited radio coverage which limit position accuracy.

Quantum accelerometers are the main component of quantum inertial navigation systems. Quantum accelerometers are self-contained systems that do not rely on any external signals (e.g., GPS, RF, etc.). Accelerometers measure how an object's velocity changes over time. A quantum accelerometer uses quantum tunneling, quantum interferometry, or other methods to measure acceleration.

However, first responder location services and situational awareness are often compromised due to one or more of: the first responders not being equipped with supportive mobile devices, the first responders not having access to a mobile application that provides reliable, real-time location, the first responders not having access to requisite technology to locate indoors, within multi-floor buildings, or within urban canyons, reliance of public safety entities/public safety agencies on siloed applications, and/or the vulnerability of GPS signals to jamming and spoofing (which can potentially disable navigation systems).

Foundation models are large-scale machine learning models trained on vast datasets, encompassing a wide range of information from text, images, audio, and more. The “foundation” aspect of these models comes from the models' ability to serve as a base for a variety of applications, enabling the models to generate new content, understand and translate languages, create realistic images, or even produce music that mimics specific styles. What sets foundation models apart is their generalizability; unlike traditional AI models that are designed for specific tasks, foundation models can adapt to a wide array of tasks with minimal additional training.

The training process of foundation models involves unsupervised or semi-supervised learning on diverse and extensive datasets, allowing the models to capture a broad understanding of natural language, visual concepts, and other data patterns. This broad understanding enables foundation models to perform tasks they were not explicitly trained for, showcasing a remarkable level of adaptability and creativity. For instance, a foundation model trained on text data can write essays, compose poetry, generate code, summarize articles, and even engage in conversation, demonstrating an understanding of context, nuance, and even humor.

Within the context of 5G networks, the adaptability of foundation models allows for a dynamic response to the unique challenges presented by this advanced technology. For example, 5G networks are designed to support large numbers of connected devices, deliver high data rates, and enable low-latency communications necessary for applications like autonomous driving and real-time gaming. Generative AI (GenAI) foundation models can analyze patterns in network traffic, device connectivity, and user demand to optimize resource allocation, manage network slices effectively, and predictively maintain network infrastructure. This results in improved network performance, enhanced user experiences, and reduced operational costs.

Moreover, GenAI foundation models can facilitate personalized user experiences by understanding and anticipating individual user needs and network usage patterns. GenAI foundation models can automate the troubleshooting process, offering real-time solutions to network issues, and interact with users, care agents, and engineers through intelligent interfaces. By leveraging natural language processing and other cognitive capabilities, GenAI foundation models can provide actionable insights, making complex network information accessible to different stakeholders. This tailored application of GenAI foundation models in monitoring and managing 5G networks illustrates a future where network operations are not only more efficient and responsive but also more aligned with the specific needs and expectations of users and operators in the 5G era.

Agent-based systems represent an innovative intersection of artificial intelligence and multi-agent systems, designed to perform complex tasks through the collaborative efforts of autonomous agents powered by foundation models. Agent-based systems leverage the capabilities of foundation models like generative ore-trained transformer (GPT)-4 to understand, process, and generate human-like text, enabling agents to communicate effectively, reason, and take actions based on the agents' environment and objectives.

At the center of an agent-based system lies a network of autonomous agents, where each agent is programmed to perform a specific role or set of tasks. These agents are powered by foundation models, which enable the agents to understand natural language instructions, process textual data, and generate contextually appropriate responses. The architecture typically includes at least a foundation model core, the agents, an environment, and a communication framework. The foundation model core comprises the central language model that provides the linguistic and cognitive capabilities for the agents. The foundation model is trained on vast datasets to understand and generate human-like text. The agents comprise autonomous entities that operate within the system. Each agent is programmed with specific functionalities, such as data collection, analysis, decision making, or communication. The environment comprises the contextual space in which the agents operate. The environment could be a virtual environment (such as a digital workspace) or a physical environment (such as a smart home or factory). The communication framework comprises the protocols and interfaces that enable the agents to interact with each other and with the large language model (LLM) foundation model core. The framework ensures seamless information exchange and coordinates actions.

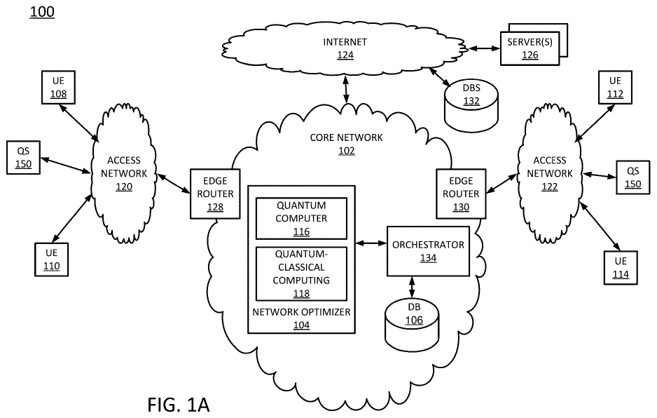

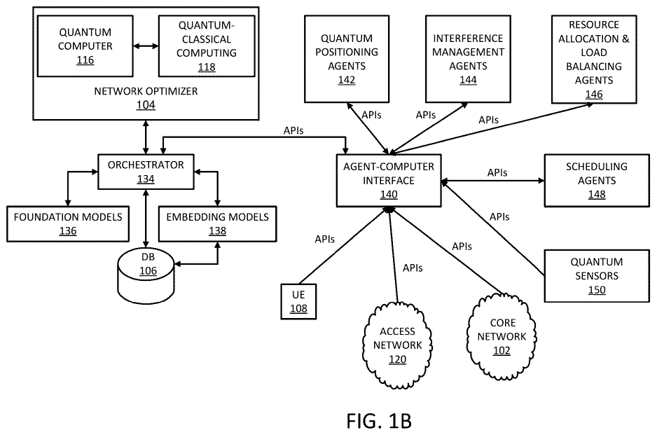

As discussed above, examples of the present disclosure utilize quantum positioning to obtain precise and real-time location data of users of communications networks (e.g., 5G, 6G, and next-generation communication networks). The location data may be used in combination with quantum computing, quantum machine learning, and agent-based systems that leverage foundation models to enhance the optimization of the communications network for resource allocation, load balancing, and interference management, resulting in increased network efficiency, improved network performance, and greater network scalability. These and other aspects of the present disclosure are discussed in greater detail below in connection with the examples of FIGS. 1-3.

To further aid in understanding the present disclosure, FIG. 1 illustrates an example system 100 in which examples of the present disclosure for optimizing fifth generation/sixth generation and next-generation networks based on quantum positioning of user devices may operate. The system 100 may comprise all or part of a 5G/6G (and/or next-generation) quantum-classical hybrid network. The system 100 may include any one or more types of communication networks, such as a traditional circuit switched network (e.g., a public switched telephone network (PSTN)) or a packet network such as an Internet Protocol (IP) network (e.g., an IP Multimedia Subsystem (IMS) network), an asynchronous transfer mode (ATM) network, a wired network, a wireless network, and/or a cellular network (e.g., 2G-5G, a long term evolution (LTE) network, and the like) related to the current disclosure. It should be noted that an IP network is broadly defined as a network that uses Internet Protocol to exchange data packets. Additional example IP networks include Voice over IP (VoIP) networks, Service over IP (SoIP) networks, the World Wide Web, and the like.

In one example, the system 100 may comprise a core network 102. The core network 102 may be in communication with one or more access networks 120 and 122, and with the Internet 124. In one example, the core network 102 may functionally comprise a fixed mobile convergence (FMC) network, e.g., an IP Multimedia Subsystem (IMS) network. In addition, the core network 102 may functionally comprise a telephony network, e.g., an Internet Protocol/Multi-Protocol Label Switching (IP/MPLS) backbone network utilizing Session Initiation Protocol (SIP) for circuit-switched and Voice over Internet Protocol (VoIP) telephony services. In one example, the core network 102 may include at least a network optimizer 104, at least one database (DB) 106, an orchestrator 134, and a plurality of edge routers 128-130. For ease of illustration, various additional elements of the core network 102 are omitted from FIG. 1.

In one example, the access networks 120 and 122 may comprise Digital Subscriber Line (DSL) networks, public switched telephone network (PSTN) access networks, broadband cable access networks, Local Area Networks (LANs), wireless access networks (e.g., an IEEE 802.11/Wi-Fi network and the like), cellular access networks, 3^{rd} party networks, and the like. For example, the operator of the core network 102 may provide a cable television service, an IPTV service, or any other types of telecommunication services to subscribers via access networks 120 and 122. In one example, the access networks 120 and 122 may comprise different types of access networks, may comprise the same type of access network, or some access networks may be the same type of access network and other may be different types of access networks. In one example, the core network 102 may be operated by a telecommunication network service provider (e.g., an Internet service provider, or a service provider who provides Internet services in addition to other telecommunication services). The core network 102 and the access networks 120 and 122 may be operated by different service providers, the same service provider or a combination thereof, or the access networks 120 and/or 122 may be operated by entities having core businesses that are not related to telecommunications services, e.g., corporate, governmental, or educational institution LANs, and the like.

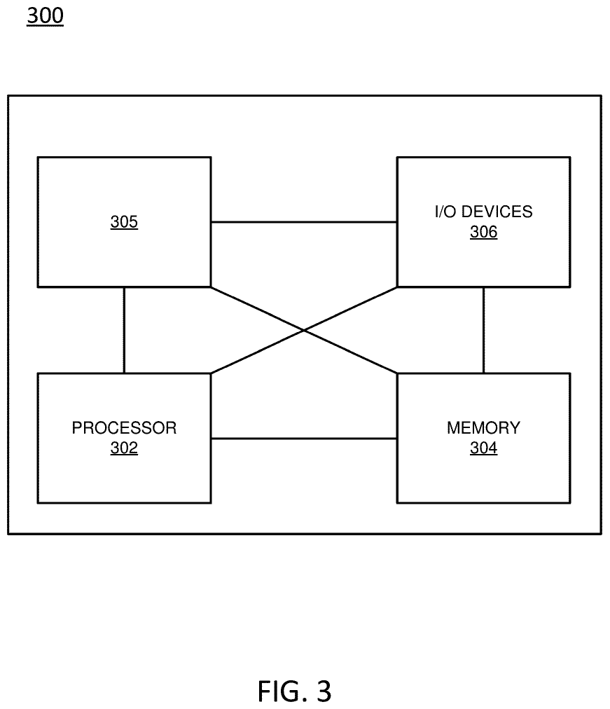

In one example, the access network 120 may be in communication with one or more user endpoint devices 108 and 110. Similarly, the access network 122 may be in communication with one or more user endpoint devices 112 and 114. The access networks 120 and 122 may transmit and receive communications between the user endpoint devices 108, 110, 112, and 114, between the user endpoint devices 108, 110, 112, and 114, the server(s) 126, the network optimizer 104, other components of the core network 102, devices reachable via the Internet in general, and so forth. In one example, each of the user endpoint devices 108, 110, 112, and 114 may comprise any single device or combination of devices that may comprise a user endpoint device, such as computing system 300 depicted in FIG. 3, and may be configured as described below. For example, the user endpoint devices 108, 110, 112, and 114 may each comprise a mobile device, a cellular smart phone, a gaming console, a set top box, a laptop computer, a tablet computer, a desktop computer, an application server, a bank or cluster of such devices, and the like.

In one example, one or more servers 126 and one or more databases 132 may be accessible to user endpoint devices 108, 110, 112, and 114 via Internet 124 in general. The server(s) 126 and DBs 132 may be associated with Internet content providers, e.g., entities that provide content (e.g., news, blogs, videos, music, files, products, services, or the like) in the form of websites (e.g., social media sites, general reference sites, online encyclopedias, or the like) to users over the Internet 124. Thus, some of the servers 126 and DBs 132 may comprise content servers, e.g., servers that store content such as images, text, video, and the like which may be served to web browser applications executing on the user endpoint devices 108, 110, 112, and 114 in the form of websites.

In accordance with the present disclosure, the network optimizer 104 may be configured to provide one or more operations or functions in connection with examples of the present disclosure for optimizing fifth generation/sixth generation and next-generation networks based on quantum positioning of user devices, as described herein. The network optimizer 104 may comprise both a quantum computer module 116 and a quantum-classical computing module 118, which may each comprise one or more physical devices, e.g., one or more computing systems or servers, such as computing system 300 depicted in FIG. 3, and may be configured as described below. Additionally, orchestrator 134 may also comprise one or more physical devices, e.g., one or more computing systems or servers, such as computing system 300 depicted in FIG. 3, and may be configured as described below. It should be noted that as used herein, the terms “configure,” and “reconfigure” may refer to programming or loading a processing system with computer-readable/computer-executable instructions, code, and/or programs, e.g., in a distributed or non-distributed memory, which when executed by a processor, or processors, of the processing system within a same device or within distributed devices, may cause the processing system to perform various functions. Such terms may also encompass providing variables, data values, tables, objects, or other data structures or the like which may cause a processing system executing computer-readable instructions, code, and/or programs to function differently depending upon the values of the variables or other data structures that are provided. As referred to herein a “processing system” may comprise a computing device including one or more processors, or cores (e.g., as illustrated in FIG. 3 and discussed below) or multiple computing devices collectively configured to perform various steps, functions, and/or operations in accordance with the present disclosure.

In one example, the network optimizer 104 may be configured to optimize fifth generation/sixth generation and next-generation communications networks. To this end, the network optimizer 104 may coordinate with the orchestrator 134 to process data received from quantum sensors 150 distributed throughout the system 100 and to orchestrate the operations of quantum agents within the system 100. Details of the network optimizer 104 and orchestrator 134, as well as other components with which the network optimizer 104 and orchestrator 134 cooperate to optimize the system 100, are discussed further below in connection with FIG. 1B.

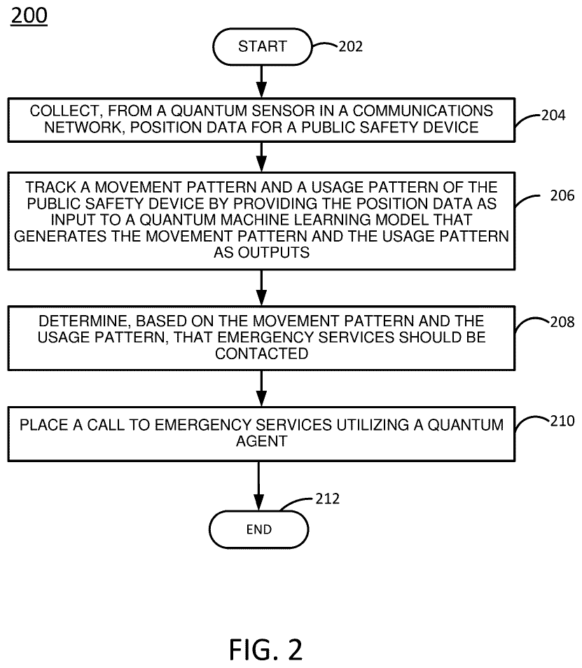

In one example, the DB 106 comprises a vector database. In one example, the DB 106 may comprise a physical storage device integrated with the orchestrator 134 (e.g., a database server or a file server), or attached or coupled to the orchestrator 134, in accordance with the present disclosure. In one example, the orchestrator 134 may load instructions into a memory, or one or more distributed memory units, and execute the instructions for optimizing fifth generation/sixth generation and next-generation communications networks, as described herein. One example method for optimizing the provision of emergency series in a communications network is described in greater detail below in connection with FIG. 2.

It should be noted that the system 100 has been simplified. Thus, those skilled in the art will realize that the system 100 may be implemented in a different form than that which is illustrated in FIG. 1, or may be expanded by including additional endpoint devices, access networks, network elements, application servers, etc. without altering the scope of the present disclosure. In addition, system 100 may be altered to omit various elements, substitute elements for devices that perform the same or similar functions, combine elements that are illustrated as separate devices, and/or implement network elements as functions that are spread across several devices that operate collectively as the respective network elements.

For example, the system 100 may include other network elements (not shown) such as border elements, routers, switches, policy servers, security devices, gateways, a content distribution network (CDN) and the like. For example, portions of the core network 102, access networks 120 and 122, and/or Internet 124 may comprise a content distribution network (CDN) having ingest servers, edge servers, and the like. Similarly, although only two access networks, 120 and 122 are shown, in other examples, access networks 120 and/or 122 may each comprise a plurality of different access networks that may interface with the core network 102 independently or in a chained manner. For example, all of the UE devices 108, 110, 112, and 114 may communicate with the core network 102 via different access networks, or only user endpoint devices 110 and 112 may communicate with the core network 102 via different access networks, and so forth. Thus, these and other modifications are all contemplated within the scope of the present disclosure.

FIG. 1B illustrates a block diagram showing the network optimizer 104 of FIG. 1A in greater detail, including connections of the network optimizer 104 to other elements of the system 100 of FIG. 1A.

As discussed above, the network optimizer 104 generally comprises a quantum computer module 116 and a quantum-classical computing module 118. The quantum computer module 116 leverages the power of quantum computing to solve complex optimization problems more efficiently than classical computing methods. In one example, the quantum computer module 116 executes quantum annealing techniques, which are a specific type of quantum computation that is well-suited for solving optimization problems. Quantum annealing works by finding the lowest energy state (or the optimal solution) of a problem encoded in a quantum system. This approach is especially effective for combinatorial optimization tasks, which are common in network management scenarios.

For instance, optimizing the allocation of network resources such as bandwidth and power levels across a dynamic and densely populated network involves evaluating numerous potential configurations to find the best configuration. Quantum annealing can explore these potential configurations simultaneously, which significantly reduces the time consumed to identify the optimal solution relative to classical methods. The ability to quickly identify an optimal configuration is crucial for maintaining high network performance and adaptability in real time.

In one example, the quantum annealing techniques executed by the quantum computer module 116 are specifically designed for operation in conjunction with machine learning applications. These quantum annealing techniques excel at finding optimal parameters in large, complex datasets, which improves the efficacy of the machine learning models.

In the network optimization context, machine learning models may be used to predict user behavior, identify patterns in network usage, and anticipate potential issues before they cause a noticeable degradation in user experience. Quantum annealing algorithms can significantly accelerate the training process of these machine learning models by efficiently searching the parameter space to determine the best-fitting parameters. The ability to efficiently determine the best-fitting parameters allows for more accurate and timely predictions, which enables a network optimization system to preemptively address network congestion, interference, and resource allocation challenges.

In the context of network optimization, quantum annealing techniques may be applied to tasks including, but not limited to: resource allocation, load balancing, and interference management. In the context of load balancing, quantum annealing may be used to dynamically allocate network resources such as bandwidth and power levels across a network. By encoding the resource allocation problem into a quantum annealing framework, the system can explore numerous potential allocations simultaneously to find the optimal distribution of resources. As an example, where multiple users with varying demands are connected to different network nodes, a quantum annealing technique can evaluate different allocations to determine the optimal allocation of bandwidth and power levels that minimizes latency and maximizes throughput for the users.

In the context of load balancing, quantum annealing techniques may be used to help balance the network load by redistributing traffic to less congested cells or frequency bands in the network. The quantum annealing techniques may evaluate multiple traffic routing options to minimize congestion and ensure a balanced load across the network. As an example, in a network that experiences fluctuating traffic patterns, a quantum annealing algorithm can identify the best routes for data packets, ensuring that no single node becomes a bottleneck while maintaining overall network efficiency.

In the context of interference management, quantum annealing techniques may be used optimize parameters for beamforming techniques to reduce interference. By considering user locations and movement patterns, a quantum annealing technique can determine the optimal angles and power levels for signal beams. Quantum annealing techniques may also be used to optimize frequency reuse by evaluating different frequency allocation strategies to minimize co-channel interference. As an example, in a dense urban environment, quantum annealing techniques may be used to dynamically adjust the beamforming parameters to ensure that signals are directed accurately, which reduces interference and improves signal quality.

Quantum annealing techniques typically involve the following phases: problem formulation, initialization, annealing, and measurement. During problem formulation, an optimization problem is formulated as an Ising model or a quadratic unconstrained binary optimization (QBUO) problem. This involves defining a cost function that needs to be minimized, which is then encoded into the Hamiltonian of the quantum system. During initialization, the quantum system is initialized in a superposition of all possible states, representing all potential solutions to the formulated problem. The initial Hamiltonian typically represents a simple problem whose ground state is easy to prepare. During annealing, the system undergoes an annealing process in which the Hamiltonian is gradually evolved from its initial state to a final state. The annealing schedule, which controls the rate of the evolution, is beneficial for ensuring that the system remains in its ground state and finds an optimal solution. During measurement, the state of the qbits, post-annealing, is measured. The resulting state represents the solution to the optimization problem. Multiple iterations of the problem formulation, initialization, annealing, and measurement may be performed to ensure the robustness of the solution.

Quantum annealing techniques may also be used to enhance machine learning models within a network optimization system, by improving model training, feature selection, and/or clustering/classification. For instance, quantum annealing may accelerate the training of machine learning models by efficiently searching for optimal parameters. This may be particularly useful for models that predict user behavior, network traffic patterns, or potential interference issues. Quantum annealing may also be used to identify the most relevant features in large datasets that contribute to accurate predictions. This may reduce the dimensionality of the datasets and improve the performance of the model. Quantum annealing may also help in clustering and classification tasks by finding the optimal grouping of data points, which is necessary for segmenting users and predicting network load distribution.

The integration of quantum annealing techniques via the quantum computer module 116 may also enhance the predictive capabilities of the network optimizer 104. For instance, quantum agents (discussed in greater detail below) may use the quantum annealing algorithms to quickly and accurately train machine learning models that predict the movement and density of users within the network. By analyzing patterns in historical and real-time data, these machine learning models can forecast areas likely to experience high traffic or interference, which may allow the network optimizer 104 to proactively adjust resources and mitigate potential issues.

Additionally, in emergency response scenarios, quantum annealing techniques may be used to optimize the deployment of first responders and resources. By predicting the most effective routes and strategies based on real-time data, quantum annealing techniques may ensure that aid reaches those in need as quickly as possible, enhancing the overall efficiency and effectiveness or emergency management.

The quantum-classical computing module 118 integrates the quantum computer module 116 with the network optimizer 104 and operates as an interface between the quantum computer module 116 and the classical components of the network optimizer to ensure seamless communication and data exchange. This hybrid approach maximizes the strengths of both quantum and classical computing. The quantum-classical computing module 118 coordinates the data flow by converting classical data into a form suitable for quantum processing and interpreting the quantum output into actionable insights for the system's agents. This ensures that the full potential of quantum computing can be harnessed to enhance network optimization.

Referring back to FIG. 1B, the network optimizer 104 is communicatively coupled to an orchestrator 134. The orchestrator 134 coordinates interactions between various components of the network optimizer 104, at least one quantum positioning agent 142, vector database 106, foundations models 136, and embedding models 138. The orchestrator 134 operates to streamline processes and manage data flow from the communications framework. The orchestrator 134 may initiate data retrieval from application programming interfaces (APIs), manage the storage and retrieval of data within the vector database 106, and ensure that data is correctly formatted and presented to the embedding models 138 for processing. The orchestrator 134 is also responsible for coordinating with the foundation models 136. Data from the APIs may be tokenized and converted to semantic vectors by the embedding models 138. The semantic vectors may then be stored in the vector database 106.

The preprocessed/tokenized data from the APIs can be converted to a format for training the foundation models 136. This requires converting the data from the APIs to a numerical tensor, which may then be provided as an input to the foundation models 136 for training. The foundation models 136 may include deep learning GPT models, which may partition the input data into smaller chunks (such as sequences of information) that can be processed in parallel to speed training.

The foundation models 136 provide the linguistic and cognitive capabilities for the quantum positioning agent 142. In one example, the foundation models 136 are trained on vast datasets to understand and generate human-like text, images, video, audio, and other signal types. The pre-trained large foundation models 136 can be fine-tuned on smaller, task-specific datasets to improve performance. Fine-tuning involves adjusting the model parameters to minimize the loss function on the smaller datasets.

Human feedback may be incorporated into the training process to align outputs of the foundation models 136 with user intent through the process of supervised fine-tuning (SFT), which adapts a pre-trained foundation model to a specific domain by fine-tuning model parameters with a labeled dataset. In one example, the domain is 5G/6G/Next-G Core and RAN data, events, messages, probe packets, and other network data. Pre-trained weights of the model can be used in this case as initial values and then updated with backpropagation on the fine-tuning dataset. This allows the foundation model to learn task-specific features while still retaining general knowledge acquired during pre-training.

The performance of the foundation models 136 can be evaluated on a validation set, which is a portion of training data set aside for evaluation purposes. Evaluation metrics such as accuracy, precision, recall, F1 scores, or other metrics may be used to compare performance of the foundation models 136 and choose the best model for a specific task.

The vector database 106 enhances the network optimizer's ability to process and utilize vast amounts of complex data efficiently. The vector database 106 stores quantum positioning data, which includes not only the latitude and longitude of users of the system 100 but also altitude, time stamps, and potentially other dimensions such as speed and direction of movement. This data is stored as multi-dimensional vectors, allowing for efficient querying and retrieval based on various criteria.

For example, when the quantum positioning agent 142 needs to determine the optimal allocation of network resources, it can query the vector database 106 to retrieve the current positions and movement patterns of users (e.g., UE 108) in a specific area. The vector database 106 can quickly return relevant data points by leveraging its efficient indexing and search capabilities. This rapid access to high-dimensional data enables the quantum positioning agent 142 to make real-time decisions, ensuring that network resources are allocated where they are needed most.

Vector databases such as vector database 106 are designed to handle high-dimensional data with high performance and scalability. Vector databases use advanced indexing techniques such as KD-trees, R-trees, and other spatial indexing methods to enable fast nearest-neighbor searches and range queries. This is particularly important in the context of the present disclosure, where a continuous stream of location data from potentially millions of users must be processed and responded to.

The scalability of vector databases ensures that the system 100 can expand to accommodate growing data volumes without degradation in performance. As more users connect to the system 100 and more data is generated, the vector database 106 can scale horizontally, distributing the data across multiple nodes and maintaining high query performance. This scalability is crucial for maintaining the efficiency and effectiveness of the network optimizer 104, especially in large-scale deployments.

In emergency response scenarios, the ability to quickly and accurately retrieve location data is important. The vector database 106 in this case may store the multi-dimensional spatial data of first responders and victims, allowing the quantum positioning agent 142 to query and visualize the disaster zone in real-time. For instance, during a natural disaster, quantum positioning agent 142 can use the vector database 106 to retrieve the current locations of all first responders and victims, visualize positions of the first responders and victims on a multi-dimensional map, and make informed decisions about resource allocation and rescue strategies.

The predictive capabilities of the network optimizer 104 are also enhanced by the vector database 106. By analyzing historical and real-time data stored in the vector database 106, quantum positioning agent 142 can predict future developments and potential risks. For example, by examining patterns in the movement of a hurricane, quantum positioning agent 142 can forecast the hurricane's path and impact areas, allowing for proactive resource allocation and evacuation planning. The vector database 106 enables the storage and retrieval of the necessary data for these predictive models, ensuring that quantum positioning agent 142 and agents 144, 146, and 148 have access to comprehensive and up-to-date information.

Beyond emergency response, vector databases are integral to the overall optimization of network performance. Quantum positioning agent 142 relies on the vector database 106 to monitor user locations and movement patterns continuously. This data informs decisions related to dynamic resource allocation, load balancing, and interference management. For example, quantum positioning agent 142 and agents 144, 146, and 148 can query the vector database 106 to identify areas with high user density and adjust network resources accordingly to prevent congestion and ensure optimal service quality.

The vector database 106 also supports the network optimizer's adaptive beamforming capabilities. By storing the precise locations and movement patterns of users, the vector database 106 allows quantum positioning agent 142 to dynamically adjust the direction and power of signal beams, minimizing interference and improving signal quality. This real-time adaptability is crucial for maintaining high performance and reliability in 5G and 6G networks.

The embedding models 138 transform various forms of data into vector representations that can be efficiently processed by the quantum techniques, quantum positioning agent 142, and other agents 144, 146, and 148. For instance, quantum positioning data, which includes spatial coordinates and temporal information, can be embedded into a lower-dimensional vector space. This transformation helps reduce the complexity of the data, making it easier to store, retrieve, and manipulate within the vector database 106 and other components of the system 100.

In one example, the embedding models 138 are trained on large datasets to learn the optimal representations for different types of data relevant to network optimization. Once trained, the embedding models 138 can be used in real-time to generate embeddings for incoming data streams. For example, as new location data is collected from users (e.g., from UE 108), the embedding models 138 can instantly convert the incoming data into vector representations that encapsulate the spatial and temporal relationships among users. The embeddings can then be used by agents 142, 144, 146, and 148 to make informed decisions about resource allocation, load balancing, and interference management.

By leveraging the embedding models 138, the network optimizer 104 can process large volumes of data more efficiently. Embeddings enable faster similarity searches and clustering operations, which are essential for identifying patterns and trends in user behavior. For example, embeddings can help quantum positioning agents 142 and an agent configured as a resource allocation and load balancing agent (e.g., agent 146 in FIG. 1B) quickly identify clusters of high user density or areas experiencing network congestion. This information is crucial for dynamic resource allocation and load balancing.

The embedding models 138 also facilitate the integration of heterogeneous data types into the decision-making process. In addition to spatial and temporal data, the system 100 might incorporate other types of information, such as network traffic patterns, user preferences, and environmental factors. The embedding models 138 can convert all of these diverse data types into a unified vector space, allowing agents 142, 144, 146, and 148 to analyze and correlate the data types effectively. This holistic approach enhances the system's ability to optimize network performance comprehensively.

The embedding models 138 therefore enhance the predictive capabilities of the network optimizer 104. By transforming historical and real-time data into embeddings, the system 100 can use machine learning algorithms to predict future trends and potential issues. For example, embeddings of historical user movement patterns and network usage can help quantum positioning agent 142 to forecast areas that might experience high traffic in the near future. This predictive insight allows agents 142, 144, 146, and 148 to preemptively adjust network resources and avoid potential congestion or service degradation.

In emergency response scenarios, the embedding models 138 enable quantum positioning agent 142 to predict the movement of first responders and victims, enhancing coordination and resource allocation. For instance, by analyzing embeddings of past disaster response data, quantum positioning agent 142 can identify optimal strategies for deploying rescue teams and medical aid. These predictive models, informed by embeddings, ensure that the system 100 can respond proactively to dynamic and evolving situations, improving overall efficiency and effectiveness.

The integration of the embedding models 138 with the quantum agent-based system further enhances the functionality and performance of the network optimizer 104. Agents 142, 144, 146, and 148 rely on embeddings to make real-time decisions based on the most relevant and concise data representations. The agent-computer interface (ACI) 140 facilitates this process by providing the agents 142, 144, 146, and 148 with the tools to generate, query, and manipulate embeddings as needed.

For example, when an agent 142, 144, 146, or 148 needs to allocate resources to a high-traffic area, the agent 142, 144, 146, or 148 can query the vector database 106 for the relevant embeddings and analyze the relevant embeddings to determine the best course of action. The ACI 140 ensures that the agents 142, 144, 146, and 148 can seamlessly access and process these embeddings, providing immediate feedback and enabling rapid adjustments. This integration ensures that the system 100 operates efficiently and effectively, even under high data volumes and complex conditions.

The ACI 140 manages communication between agents 142, 144, 146, and 148, orchestrator 134, and other components through a robust framework that includes standardized protocols, real-time data exchange, and intelligent coordination. The ACI 140 ensures seamless interaction, synchronized actions, and adaptive decision-making, enabling the network to dynamically adjust resources and maintain optimal performance. The ACI 140 uses RESTful APIs for communication between UEs (e.g., UEs 108, 110, 112, and 114), the RAN (e.g., access networks 120 and 122), the core network 102, agents 142, 144, 146, and 148, orchestrator 134, and other components, providing a standardized interface for data exchange and command execution.

In one example, the ACI 140 standardizes data interchange formats using JavaScript Object Notation (JSON) and eXtensible Markup Language (XML), ensuring compatibility and ease of parsing across different systems. In a further example, the ACI 140 may employ protocol buffers (Protobuf), which provide compact, efficient, and language-neutral data interchange.

When an agent 142, 144, 146, or 148 is initialized, the agent 142, 144, 146, or 148 registers with the ACI 140 by providing its capabilities and endpoint information. The ACI 140 may maintain a registry of all active agents 142, 144, 146, and 148, facilitating discovery and interaction. In one example, the ACI 140 implements a publish-subscribe messaging model where agents 142, 144, 146, and 148 can subscribe to specific data streams or events. When an event occurs or new data is available, the ACI 140 may publish information about the event or new data to the subscribed agents 142, 144, 146, and 148. In a further example, the ACI 140 supports direct messaging channels for direct, low-latency communication between agents 142, 144, 146, and 148. Direct, low-latency communications facilitate time-sensitive coordination tasks, such as real-time traffic management.

In one example, the ACI 140 may use a task scheduler to coordinate the actions of multiple agents 142, 144, 146, and 148. Tasks may be prioritized based on their urgency and impact on network performance. In a further example, the ACI 140 may employ an event-driven architecture to handle asynchronous events, ensuring that agents 142, 144, 146, and 148 can respond promptly to changes in network conditions. The ACI 140 may maintain the state of each of the agents 142, 144, 146, and 148, including current tasks, status, and last known data. This state information helps to ensure synchronized actions and avoid conflicts.

In one example, the ACI 140 interfaces with network components such as routers, switches, and base stations using standard APIs (e.g., SNMP, NETCONF) and communication protocols. This ensures seamless integration and control over the network hardware. The ACI 140 may send commands to network devices to adjust settings based on the decisions made by agents 142, 144, 146, and 148. These commands include changes to routing paths, bandwidth allocation, and power levels.

In one example, the ACI 140 manages real-time data streams from various sources, ensuring that all components have access to the latest information. The real-time data streams may include traffic data, user locations, signal strengths, and environmental conditions. The ACI 140 may ensure that all components operate with consistent and synchronized data, preventing discrepancies and ensuring accurate decision-making.

The ACI 140 may use encryption protocols (e.g., TLS) to secure communication channels between agents 142, 144, 146, and 148, orchestrator 134, and other network components. In one example, the ACI 140 implements authentication mechanisms to verify the identities of agents 142, 144, 146, and 148, and orchestrator 134, ensuring that only authorized entities can execute commands and access data.

In one example, the ACI 140 continuously monitors key performance metrics such as latency, throughput, and error rates. The continuously monitored metrics are used to assess the impact of resource adjustments and optimize network performance. The ACI 140 may establish a continuous feedback loop, where the ACI 140 receives real-time feedback from agents 142, 144, 146, and 148, and orchestrator 134 on the effectiveness of the ACI's actions. This feedback is used to refine algorithms and improve decision-making.

The agents 142, 144, 146, 148 comprise autonomous entities that operate within the system 100. Each agent 142, 144, 146, and 148 is equipped with specific functionalities, such as data collection, analysis, decision-making, or communication. In one example, the agents 142, 144, 146, and 148 include quantum positioning agents 142, interference management agents 144, resource and load balancing agents 146, and scheduling agents 148. In other examples, however, other types of agents may be included or substituted. Agents 142, 144, 146, and 148 use the foundation models 136 to understand instructions, queries, and contextual information. In one example, agents 142, 144, 146, and 148 may run on classical computer hardware. However, in other examples, agents 142, 144, 146, and 148 may run on quantum computers.

In one example, the quantum positioning agents 142 collect quantum positioning data from 5G/6G/Next-G quantum sensors (QS) 150 to achieve superior precision compared to traditional positioning systems such as GPS. The quantum positioning data enhances various aspects of the network, including resource allocation, load balancing, interference management, and public safety applications.

In one example, the interference management agents 144 leverage quantum positioning data, agent-based systems, and advanced quantum computing to minimize wireless interference, enhance wireless signal quality, and ensure efficient utilization of network resources. Interference management helps to maintain high data rates and low latency.

In one example, an adaptive beamforming process within the interference management agents 144 uses precise location data provided by the quantum positioning. By knowing the exact positions of users (e.g., UEs 108, 110, 112, and 114), the system 100 can dynamically adjust the direction and power of wireless signal beams. This targeted approach minimizes interference by directing signals specifically towards the intended users while avoiding areas where interference with other signals might occur. The interference management agents 144 aid this process by continuously monitoring user locations and network conditions. The interference management agents 144 utilize the ACI 140 to communicate real-time adjustments such as beam forming parameters, ensuring optimal signal delivery and reduced interference.

For instance, in one example, the interference management agents 144 may manage interference among a plurality of phased array antennas. A phased array antenna comprises a plurality of individual antenna elements that can independently adjust the phase and amplitude of the signals the antenna elements transmit and receive. Each antenna element is equipped with a phase shifter that can alter the phase of a signal transmitted or received. By precisely controlling these phase shifts, the phase array antenna can steer the direction of the signal beam.

In one example, the interference management agents 144 may use digital signal processing techniques to control the phase and amplitude of the signals at each antenna element, which allows for precise beam steering and shaping. Adaptive techniques (e.g., least mean squares, recursive least squares, etc.) may be implemented to continuously adjust the beamforming parameters based on real-time data. The radiation pattern of the phased array antenna may be adjusted to create null points in the direction of interference sources, reducing the impact of these interference sources on the desired signals.

In one example, the interference management agents 144 may initialize a beamforming technique with the current location data and signal quality metrics, setting the initial phase and amplitude for each antenna element of a phased array antenna. As new location and signal quality metrics are received, the phase and amplitude settings for the antenna elements may be adjusted to maintain optimal beam direction and shape. Continuous monitoring of signal quality metrics may provide feedback that can be used to ensure that the beamforming adjustments are effective.

In another example, the interference management agents 144 may perform real-time adjustment of signal beams from phase array antennas. For instance, the movements of users and devices (e.g., UEs 108, 110, 112, and 114) may be continuously tracked to ensure that the signal beams are always directed toward the current locations of the users and devices. The beamforming parameters of the signal beams may also be adjusted to adapt to environment changes, such as moving obstacles or varying weather conditions.

In another example, the interference management agents 144 may dynamically adjust the power levels of the signal beams, based on the distance to the user(s) and the presence of interference. Higher power levels may be implemented to serve distance users or in environments experiencing greater amounts of interference. Lower power levels may be implemented to users who are close by or in environments where interference is minimal. Interference management agents 144 may determine optimal power levels for each signal beam based on factors including user density, signal quality, and environmental conditions.

In another example, the interference management agents 144 may perform frequency coordination. By considering the exact positions of users, frequency reuse patterns can be optimized. This involves dynamically allocating frequencies to different users and areas to minimize co-channel interference, where signals from different cells overlap and cause degradation in performance. Interference management agents 144 may analyze real-time and historical data to predict potential interference scenarios and proactively adjust frequency allocations. Through the ACI 140, interference management agents 144 can swiftly update frequency settings and maintain optimal network performance, even in densely populated urban environments where interference risks are higher.

In one example, interference management agents 144 communicate using machine learning techniques and foundation models 136 to predict and mitigate interference issues. Interference management agents 144 continuously assess network conditions, user distribution, and signal quality to identify potential interference hotspots. The ACI 140 facilitates efficient communication between interference management agents 144, allowing interference management agents 144 to share data and coordinate actions seamlessly. This collaborative approach ensures that interference management decisions are informed by the most up-to-date and comprehensive data available. For example, if an interference management agent 144 detects increasing interference in a particular cell, the interference management agent 144 can alert other interference management agents 144 and collectively implement strategies to mitigate the issue, such as adjusting beamforming angles or reallocating frequencies.

The ACI 140 incorporates robust error handling and feedback mechanisms to maintain high accuracy and efficiency. Interference management agents 144 receive immediate feedback on their actions, such as syntax checking during configuration edits, ensuring that changes are correctly implemented. The ACI 140 also provides concise and relevant context to interference management agents 144, helping the interference management agents 144 make informed decisions without being overwhelmed by extraneous information. The streamlined ACI 140 reduces the risk of errors and enhances the overall effectiveness of the interference management agents 144.

By combining quantum positioning data, agent-based decision-making, and advanced quantum computing, the interference management agents 144 effectively reduce interference, improve signal quality, and ensure efficient use of network resources. The integration of the ACI 140 enhances the capabilities of interference management agents 144, enabling the interference management agents 144 to perform complex interference management tasks with high accuracy and efficiency.

In one example, the resource and load balancing agents 146 allocate and load balance resources, including wireless bandwidth and power levels which are dynamically adjusted based on the exact location and density of devices (e.g., UEs 108, 110, 112, and 114). Quantum positioning data is used to determine the precise locations of devices, enabling real-time adjustments to optimize network performance. Quantum machine learning techniques process the quantum positioning data to dynamically allocate and load balance resources by considering factors such as user density, current network load, and predicted device movement.

Resource allocation and load balancing agents 146 continuously monitor real-time network conditions and user locations and use foundation models 136 to predict future resource needs. Resource allocation and load balancing agents 146 communicate with each other to dynamically adjust network resources via ACI 140. Resource allocation and load balancing agents 146 also identify congested areas and coordinate with each other via ACI 140 to redirect devices in a way that balances the load across the network. Resource allocation and load balancing agents 146 use the ACI 140 to efficiently search for and access relevant network configuration files and data. Resource allocation and load balancing agents 146 also view and edit network configurations using ACI 140 to receive immediate feedback on changes.

For example, quantum positioning may indicate a high concentration of devices in a particular area. Resource allocation and load balancing agents 146 may detect this high concentration of devices and call via the ACI 140 quantum machine learning techniques to increase the available wireless bandwidth by adjusting power levels for wireless nodes (5G/6G/Next-WIFI, etc.) in the particular area to ensure adequate service quality. Network traffic is load balanced by redirecting at least some of the devices to less congested nodes and/or frequency bands based on precise locations.

In another example, edge compute and network resources may be strategically deployed based on user concentrations. By using precise location data, resource allocation and load balancing agents 146 may position or re-position edge compute and network servers closer to users, reducing latency and improving service quality. Resource allocation and load balancing agents 146 could also position mobile edge and network resources such as cells on wheels, aerial drones/aircraft and satellite resources.

In another example, a large event or public safety emergency may be identified by quantum positioning data and quantum machine learning techniques that predict hotspots where user demand is high or predicted to be high. Resource allocation and load balancing agents 146 may allocate edge compute and network resources to these hotspots to ensure faster processing and reduce latency.

In another example, detailed location data and device movement patterns may be analyzed using quantum machine learning models to predict future movement patterns and resource demand. Resource allocation and load balancing agents 146 may increase RAN and general network resource capacity to preemptively accommodate future demand.

In one example, the scheduling agents 148 manage workflow between various agents 142, 144, 146, and 148 within the system 100. Using dynamic scheduling, real-time monitoring, and adaptive feedback mechanisms, the scheduling agents 148 may optimize the performance and responsiveness of the agents 142, 144, 146, and 148. The scheduling agents 148 may identify and categorize tasks, assign priorities, allocate resources, and ensure efficient inter-agent communication.

In one example, the scheduling agents 148 identify tasks based on incoming data and system requirements. These tasks can include data collection, resource allocation, interference management, and optimization computations. Tasks may be categorized based on their type (e.g., monitoring, control, prediction) and priority (e.g., high-priority tasks for emergency scenarios, low-priority tasks for routine maintenance). When faced with a complex task, the scheduling agents 148 may use reasoning to break the complex task down into smaller, more manageable subtasks. This process allows the scheduling agents 148 to approach the complex task systematically, tackling each component individually before integrating the results into a comprehensive solution.

Tasks may be assigned priority levels based on their urgency and importance. High-priority tasks may include tasks that have immediate impact on network performance or user experience, such as interference mitigation during peak hours or emergency communication during disasters. The scheduling agents 148 may dynamically adjust task priorities based on real-time conditions. For example, if a sudden spike in traffic is detected, tasks related to load balancing and resource allocation may be given higher priority.

In one example, the scheduling agents 148 determine when and how to use various tools. Determining when and how to use the tools may include deciding which APIs to call, what external resources to access, or which specialized functions to employ to accomplish specific subtasks. Advanced reasoning capabilities may allow the scheduling agents 148 to consider the long-term consequences of actions and decisions. Considering the long-term consequences may involve projecting potential outcomes, evaluating risks, and choosing the most appropriate course of action based on these projections.

In one example, the scheduling agents 148 may continuously monitor key performance metrics such as task completion times, resource utilization, and inter-agent communication latency. The monitored metrics may be used to assess the efficiency and effectiveness of the workflow management. The scheduling agents 148 may also track the progress of ongoing tasks to ensure the ongoing tasks are on schedule and may identify any delays or issues that need to be addressed.

In another example, the scheduling agents 148 may establish a feedback loop by which agents 142, 144, 146, and 148 provide feedback on task execution and resource allocation. This feedback may be used by the scheduling agents 148 to refine scheduling algorithms and improve future task management. In one example, the scheduling agents 148 can learn from past scheduling decisions and adapt approaches to improve performance over time. This may require updating internal models, refining decision-making processes, updating resource allocation or expanding knowledge bases. For example, if certain tasks consistently require more resources than initially allocated, the scheduling agents 148 may adjust future allocations accordingly.

In the context of emergency response, environmental sensors may detect a natural disaster, triggering an emergency response. The scheduling agents 148 may prioritize tasks related to emergency communication and resource deployment.

Quantum sensors 150 leverage the principles of quantum mechanics. According to examples of the present disclosure, quantum sensors 150 are widely distributed throughout 5G/6G/Next-G wireless networks. This distribution of quantum sensors 150 allows for wide area sensing capabilities across 5G/6G/Next-G networks and the potential for novel sensing modalities that exploit quantum phenomena. In one example, the quantum sensors 150 may include any one or more of the following: quantum gravimeters, quantum accelerometers, quantum clocks, and quantum satellites. In a further example, the quantum sensors may be integrated with other devices, including Internet of Things (IoT) devices, drones, connected vehicles, satellites, and other devices.

Quantum gravimeters measure tiny variations in the Earth's gravitational field, which can be used to determine precise altitude and location. Quantum gravimeters are highly sensitive and can detect minute changes, providing more accurate positioning data. Quantum gravimeters can achieve precision on the order of a few micro-Galileos (μGal, where 1 Gal=1 cm/s²). Some advanced systems claim precision as high as 0.1-1 μGal. The absolute accuracy of quantum gravimeters can be in the range of 1-10 μGal, depending on the specific instrument and calibration methods. Quantum gravimeters often show excellent long-term stability, with drift rates much lower than traditional spring-based gravimeters. Compared to classical instruments, quantum gravimeters have been shown to be ten to one hundred times more precise than the best classical gravimeters.

Quantum accelerometers measure acceleration with high precision, to track the movement and velocity of objects. Quantum accelerometers can maintain accuracy over longer periods and distances compared to classical accelerometers. Quantum accelerometers can achieve precision on the order of nano-g to pico-g (1 g=9.81 m/s²), depending on the specific implementation and measurement time. The absolute accuracy of quantum accelerometers can be in the range of micro-g to nano-g, again depending on the specific instrument and calibration methods. Quantum accelerometers typically exhibit excellent long-term stability, with very low drift rates compared to classical MEMS or mechanical accelerometers. While often optimized for high precision at lower frequencies, some quantum accelerometers can operate over bandwidths of several kHz. Quantum accelerometers can maintain their high precision over a wide dynamic range, often spanning several orders of magnitude. Compared to classical accelerometers, and depending on the application, quantum accelerometers have been shown to be one hundred to one thousand times more precise than high-end classical accelerometers.

Quantum clocks are ultra-precise clocks based on quantum mechanics to ensure accurate timekeeping, which is useful for synchronization in positioning systems. Quantum clocks reduce timing errors that can lead to inaccuracies in location data. The best quantum clocks can achieve accuracy on the order of 10⁻¹⁸ to 10⁻¹⁹. Quantum clocks are approximately one hundred times more accurate than the best cesium fountain clocks, which were the previous standard. Short-term stability can be as good as 10⁻¹⁶ over a few seconds, and long-term stability can reach 10⁻¹⁸ over days or weeks. The precision of quantum clocks can be in the range of attoseconds (10⁻¹⁸ seconds).

Satellites equipped with quantum sensors can provide global positioning data, which ensures accurate location tracking even in remote or challenging environments. Integration of quantum satellite-based networks can thus extend the coverage and reliability of a positioning system. Quantum satellite 5G/6G/Next-G networks offer redundant paths for data transmission, which ensures that location data remains available even if ground-based infrastructure fails.

FIG. 2 illustrates a flowchart of an example method 200 for optimizing the provision of emergency series in a communications network, in accordance with the present disclosure. In one example, steps, functions and/or operations of the method 200 may be performed by a device as illustrated in FIG. 1, e.g., network optimizer 104, orchestrator 134, or any one or more components thereof. In another example, the steps, functions, or operations of method 200 may be performed by a computing device or system 300, and/or a processing system 302 (e.g., having at least one processor) as described in connection with FIG. 3 below. For instance, the computing device 300 may represent at least a portion of the network optimizer 104 or orchestrator 134 in accordance with the present disclosure. For illustrative purposes, the method 200 is described in greater detail below in connection with an example performed by a processing system, such as processing system 302.

The method 200 begins in step 202 and proceeds to step 204. In step 204, the processing system may collect, from a quantum sensor in a communications network, position data for a public safety device.

In one example, the public safety device may comprise an automated external defibrillator (AED), a fire extinguisher, an epinephrine auto-injector, an inhaler, a fire alarm, a carbon monoxide detector, a mobile communications device (e.g., a smart phone, a tablet computer, or the like), a medical monitoring device (e.g., a pacemaker, a blood glucose monitor, a fall sensor, or the like), an IoT-enabled medical device, or another type of environmental sensor. In one example, the public safety device may be equipped with a quantum sensor that is in communication with the processing system, e.g., via an agent-computer interface that allows the quantum sensor, as well as other quantum sensors and quantum agents, to communicate with the processing system.

In one example, the public safety device may have a default position, and the processing system may determine, based on the position data, that the public safety device has been moved from the default position. The default position may be defined by a latitude coordinate, a longitude coordinate, and an altitude coordinate. The altitude coordinate may help to identify the default position within a multistory building such as a hospital, an office building, or the like.

In one example, the position data may comprise a stream of physical positions or locations of the public safety device, where each physical position or location may be timestamped. In one example, the position data may include a latitude coordinate, a longitude coordinate, and an altitude coordinate to define each physical position or location in the stream.

In step 206, the processing system may track a movement pattern and a usage pattern of the public safety device by providing the position data as input to a quantum machine learning model that generates the movement pattern and the usage pattern as an output.

In one example, the quantum machine learning model may be executed by a quantum computing module or a quantum-classical computing module in the communications network. The quantum machine learning model may be trained to take as input a set of position data from a quantum sensor and to track movement of the quantum sensor (or a device associated with the quantum sensor, such as a public safety device) based on the set of position data.

The quantum machine learning model may be further trained to predict, based on the set of position data and/or other data provided by the quantum sensor, a usage pattern of a device associated with the quantum sensor (e.g., a public safety device to which the quantum sensor is attached). For instance, the quantum sensor may provide information about the device such as power level or other information from which the quantum machine learning model may be able to predict whether the device has been used (e.g., whether an automated external defibrillator or an epinephrine auto-injector has been used). For instance, the quantum machine learning model may be able to infer that the public safety device has been used based on a power or battery level of the public safety device dropping, or based on a sudden surge in power consumed or utilized by the public safety device. The quantum sensor may also provide readings from the public safety device, such as carbon monoxide levels, blood glucose measurements, smoke levels, or the like.

In step 208, the processing system may determine, based on an analysis of the movement pattern and the usage pattern, that emergency services should be contacted.

For instance, based on an analysis of the movement pattern and the usage pattern, the processing system may be able to infer whether the public safety device has been moved from its default position, as well as whether the public safety device has been used since being moved from its default position. For instance, the processing system may be able to determine that a fire extinguisher has been moved to a room that is down the hall from the fire extinguisher's default position, and that the fire extinguisher has been used. This may indicate that a fire has occurred (or is occurring) in the vicinity of the fire extinguisher's current position.

In step 210, the processing system may place a call to emergency services utilizing a quantum agent.

In one example, the processing system may engage a quantum agent to place a call (e.g., a voice call, a video call, a text message, or the like), using a communications device, to emergency services (e.g., a 911 service requesting emergency assistance from law enforcement personnel, firefighting personnel, and/or Emergency Medical Technician (EMT) personnel). In a further example, the processing system may engage a quantum agent to generate information about the usage of the public safety device in a manner that can be understood by one or more emergency services. For instance, large language models and/or speech generation/synthesis technology may be used to generate a synthesized audio output to provide to one or more emergency services.

The call to emergency services may include information about the usage of the public safety device, such as the type of public safety device, the current location of the public safety device, a time at which the public safety device was used, a position of the public safety device at the time that public safety device was used, readings measured by the public safety device, and/or other information. In one example, the processing system (or another device with which the processing system is in communication) may plot position data of the public safety device on a 3D map. For instance, the plotted position data may include the current location of the public safety device or a position of the public safety device at the time that public safety device was used. This may help emergency services to not only identify the precise location (e.g., floor and/or room of a multistory building) at which assistance is needed, but also to plan the safest and/or most efficient way to approach the location at which assistance is needed. The method 200 may end in step 212.

It should be noted that the method 200 may be expanded to include additional steps or may be modified to include additional operations with respect to the steps outlined above. In addition, although not specifically specified, one or more steps, functions, or operations of the method 200 may include a storing, displaying, and/or outputting step as required for a particular application. In other words, any data, records, fields, and/or intermediate results discussed in the method can be stored, displayed, and/or outputted either on the device executing the method or to another device, as required for a particular application. Furthermore, steps, blocks, functions or operations in FIG. 2 that recite a determining operation or involve a decision do not necessarily require that both branches of the determining operation be practiced. In other words, one of the branches of the determining operation can be deemed as an optional step. Furthermore, steps, blocks, functions or operations of the above described method can be combined, separated, and/or performed in a different order from that described above, without departing from the examples of the present disclosure.

By integrating quantum sensors and quantum satellite-based 5G/6G/Next-G networks, the precision, reliability, and resilience of the 5G/6G/Next-G network optimizer can be greatly enhanced. Applications in resource allocation, load balancing, interference management, and public safety demonstrate its beneficial role in improving network performance and ensuring effective response during emergencies.

For instance, accurate and real-time location data allows the disclosed system to dynamically allocate 5G/6G/Next-G network resources based on user density and movement patterns. For example, an accident on a major vehicle expressway may cause vehicle traffic to slow down and back up. The network can adjust for more traffic in the concentrated area around the accident. Accurate and real-time location data may be used to dynamically allocate 5G/6G/Next-G network resources based on user density and movement patterns. Bandwidth and power levels may be adjusted in real-time to optimize network performance and ensure efficient resource use.

By knowing the precise locations and movements of users, the disclosed system can distribute 5G/6G/Next-G network traffic more evenly, preventing congestion in high-density areas and improving overall network efficiency.

Quantum positioning agents may enable precise beamforming techniques by providing exact user locations. This minimizes interference and enhances signal quality by directing signals accurately to the intended recipients. Accurate positioning data allows for optimized frequency reuse, reducing co-channel interference and improving overall 5G/6G/Next-G network performance.

Quantum positioning data enhances the coordination and effectiveness of emergency response efforts. First responders can be tracked in real-time, ensuring that resources are deployed efficiently, and that 5G/6G/Next-G communication remains robust during emergencies. In disaster scenarios, quantum positioning agents can provide important data for coordinating rescue operations, monitoring environmental conditions, and managing evacuations. The high precision and real-time updates ensure that decisions are based on the most accurate information available. During a natural disaster, such as an earthquake, quantum positioning agents may continuously track the locations of first responders and victims using quantum sensors and satellites. The location data may be fed into the 5G/6G/Next-G network optimizer, which may use the location data to allocate resources dynamically, direct rescue teams, and manage communication channels. The disclosed system can quickly adapt to changing conditions, ensuring that first responders receive real-time updates and guidance, which enhances their effectiveness and safety.

By integrating quantum sensors and quantum satellite-based 5G/6G/Next-G networks, the precision, reliability, and resilience of the 5G/6G/Next-G network optimizer can be greatly enhanced. Applications in resource allocation, load balancing, interference management, and public safety demonstrate its beneficial role in improving network performance and ensuring effective response during emergencies.

In emergency scenarios, the ability to accurately locate and efficiently communicate with first responders is paramount. The disclosed system leverages advanced quantum positioning data to provide real-time, highly accurate location information. This enhanced locational accuracy is crucial for coordinating rapid and effective responses during crises such as natural disasters, terrorist attacks, or large-scale accidents. By integrating quantum positioning data with robust communication frameworks like the FirstNet communication framework, the disclosed system can ensure that first responders receive timely and precise information, enabling the first responders to make informed decisions and act swiftly.

The agent-based implementation disclosed herein significantly improves the system's effectiveness in managing emergencies. Quantum agents, equipped with advanced techniques and foundation models, can continuously monitor the locations of first responders and victims. The quantum agents may use quantum positioning data to track movements and positions with high precision, ensuring that response teams are always aware of the current situation. These quantum agents coordinate with each other via the ACI, a sophisticated tool that facilitates seamless communication and data sharing. The ACI allows quantum agents to access relevant files, update configurations, and receive immediate feedback on actions, ensuring that the quantum agents can operate efficiently and effectively.

A feature of the disclosed system's emergency response capabilities is the creation of a multi-dimensional map that incorporates various spatial dimensions, including latitude, longitude, altitude, and time. This multi-dimensional map provides a comprehensive view of the disaster area, allowing quantum agents to visualize the situation in real-time. The spatial dimensions enable quantum agents to identify not only the horizontal and vertical positions of first responders and victims but also the movements of the first responders and victims over time. This dynamic mapping capability is crucial for understanding the evolving nature of emergencies and for planning effective response strategies. Quantum agents may use this multi-dimensional map to pinpoint areas with the highest concentration of victims, identify safe routes for rescue teams, and mark hazardous zones that need to be avoided.

In addition to real-time tracking and mapping, the disclosed system incorporates advanced prediction capabilities. Quantum agents use quantum machine learning models to analyze historical and real-time data, enabling the quantum agents to forecast future developments and potential risks. For instance, in the case of an ongoing natural disaster like a hurricane, quantum agents can predict the storm's path, intensity, and potential impact areas. These predictions help in proactive resource allocation and strategic planning. Quantum agents can anticipate the needs for medical aid, evacuation, and other important resources, ensuring that these resources are deployed where they will be most effective.

For instance, during a natural disaster, such as an earthquake or a hurricane, the need for coordinated rescue efforts is immediate and critical. Quantum agents may use quantum positioning data to track the precise locations of first responders and victims in real-time. The location data allows the quantum agents to create an accurate, multi-dimensional map of the disaster zone, highlighting areas with the highest concentration of victims and identifying safe routes for rescue teams. Through FirstNet or a similar communication framework, the quantum agents can coordinate with emergency services, ensuring that all responders are aligned and informed.

For example, if the quantum agents detect that a particular area has a high number of trapped individuals, the quantum agents can prioritize sending rescue teams to that location. The quantum agents can also identify safe zones for setting up medical aid stations and command centers. The quantum agents, utilizing the ACI, may continuously update their data and strategies based on new information, such as aftershocks or changing weather conditions. This real-time adaptability is important for maintaining an effective response.

Moreover, the integration of FirstNet or a similar communication framework ensures that communication remains robust even in challenging conditions. FirstNet provides a dedicated network for public safety communications, which is less likely to be congested or disrupted during emergencies. Quantum agents may use this network to share data, coordinate actions, and provide updates, ensuring that all first responders have access to the information they need. The ACI may facilitate this process by allowing the quantum agents to efficiently manage and disseminate information, reducing the risk of miscommunication and ensuring that everyone is working towards the same objectives.

In addition, predictive models can help prepare for and respond to emergencies. Quantum agents may analyze patterns in data to forecast potential risks and developments. For instance, in the event of an ongoing hurricane, the quantum, agents can predict the trajectory and speed of the storm, allowing the quantum agents to pre-position resources and evacuation plans. This foresight enables a more proactive approach to disaster management, minimizing the impact on affected communities and improving overall response effectiveness.

The integration of quantum location data, advanced quantum computing, and agent-based systems within the network optimization framework also has numerous Internet of Things (IoT) public safety applications. These IoT public safety applications improve the accuracy and efficiency of emergency response, disaster management, traffic control, surveillance, health monitoring, environmental detection, and automated drone operations. These IoT public safety applications leverage precise real-time location data, efficient resource allocation, and enhanced communication capabilities to improve emergency response, disaster management, and overall public safety.

IoT-enabled emergency response systems can significantly benefit from quantum location data and the network optimization system. These IoT-enabled emergency response systems use a network of connected devices, such as sensors, cameras, and wearable devices, to provide real-time data on the location and condition of first responders and victims.

For instance, during a natural disaster, IoT devices equipped with quantum sensors can provide real-time data on the exact locations of victims and first responders. This location data can be processed by the network optimization system, allowing quantum agents to dynamically allocate resources, optimize rescue routes, and ensure efficient communication through FirstNet or a similar communication framework. First responders equipped with wearable IoT devices may receive real-time updates and guidance, thereby improving their effectiveness and safety.

Furthermore, medical and public safety devices, such as automated external defibrillators (AEDs), fire extinguishers, epinephrine auto-injectors, inhalers, fire alarms, carbon monoxide detectors, and other essential equipment, play a pivotal role in emergency situations, often within indoor environments. Accurate location reporting is important for ensuring timely and effective assistance. Traditional methods often lack the precision necessary for complex structures, such as multi-story buildings.

The disclosed system can be used to leverage quantum location technology to report the precise latitude, longitude, and altitude of a medical or public safety device when the device is removed from its station. This accurate location data can be used to enhance emergency response efficiency, particularly in complex indoor environments. When a device, such as an AED, is taken from its station, the disclosed system may use quantum location technology to provide detailed coordinates (latitude, longitude, and altitude). These coordinates enable precise identification of the device's location within complex building structures. Artificial intelligence models may be used to detect the movement of these devices and analyze the devices' usage patterns. Based on this analysis, the disclosed system can determine whether an emergency call to 911 or other relevant emergency services is warranted. If necessary, the disclosed system may utilize large language models (LLMs) and speech generation technology to report the situation during the emergency call. The disclosed system can also create a detailed 3D map of the building. By integrating this 3D map with a device's precise coordinates, machine learning techniques can predict the exact room number in a multistory building where an emergency incident is occurring. This is particularly useful for locating patients or identifying the exact location of a fire or other emergencies within a building.

In another example, IoT devices deployed in disaster-prone areas can continuously monitor environmental conditions and provide early warnings for events such as earthquakes, floods, or wildfires. The integration of quantum location data enhances the accuracy and reliability of these systems. For instance, IoT quantum sensors placed in seismic zones can detect early signs of an earthquake. Quantum location data ensures precise positioning of these IoT quantum sensors, thereby improving the accuracy of earthquake detection and prediction. The disclosed system may process this location data, enabling quantum agents to coordinate early warnings, evacuations, and resource deployments. Real-time communication with IoT-enabled infrastructure, such as automated barriers and traffic control systems, ensures smooth and safe evacuations.

In another example, IoT applications in traffic management can leverage quantum location data to improve the safety and efficiency of transportation networks during emergencies. For instance, during a major city-wide emergency, such as a terrorist attack or a large-scale accident, IoT-enabled traffic management systems can use quantum location data to monitor vehicle movements and congestion in real-time. The disclosed system may dynamically adjust traffic signals, route emergency vehicles, and provide real-time traffic updates to citizens through connected vehicles and mobile devices. This ensures that emergency responders can reach affected areas quickly while minimizing disruption to the general public.

In another example, quantum location data can enhance the capabilities of IoT-based surveillance systems in smart cities, providing more accurate tracking and monitoring of public spaces. For instance, IoT cameras and sensors deployed throughout a city can use quantum location data to provide precise monitoring of public spaces. In the event of a security threat, such as an active shooter or a public disturbance, the disclosed system can quickly identify and track the suspect's movements. Quantum agents use this movement data to coordinate with law enforcement, deploy resources, and provide real-time updates to citizens through connected devices. This ensures a swift and coordinated response to security threats.

In another example, IoT devices for health monitoring can be integrated with quantum location data to provide better assistance and care during emergencies. For instance, wearable health monitors equipped with quantum positioning sensors can track the location and vital signs of individuals (e.g., the elderly or individuals with chronic conditions). In an emergency, such as a fall or a sudden health crisis, the disclosed system can instantly locate the individual and dispatch medical responders. The disclosed system can also communicate with nearby IoT-enabled medical devices to provide remote diagnostics and treatment instructions, thereby improving the chances of a positive outcome.

In another example, quantum location data can enhance the accuracy of IoT-based environmental monitoring systems, thereby providing early detection of hazards such as chemical spills, radiation leaks, or pollution. For instance, IoT sensors deployed in industrial areas can monitor for hazardous chemical leaks. Quantum location data ensures precise positioning of these IoT sensors, enhancing the accuracy of leak detection. When a leak is detected, the disclosed system may process the data from the IoT sensors, alerting authorities and coordinating the evacuation of affected areas. The system can also communicate with IoT-enabled containment systems to mitigate the hazard, ensuring public safety.

In another example, IoT-enabled drones equipped with quantum positioning systems can be used for various public safety applications, including search and rescue, surveillance, and delivery of medical supplies. For instance, in a search and rescue operation following a natural disaster, IoT-enabled drones can use quantum location data to navigate and search affected areas with high precision. The disclosed system may coordinate the drones' movements, ensuring the drones cover the necessary areas efficiently. Drones can provide real-time video feeds, deliver medical supplies, and assist in locating trapped victims, thereby significantly enhancing the effectiveness of the rescue operation.

In another example, quantum location data can enhance the resilience and efficiency of IoT-enabled emergency communication networks. For instance, in the aftermath of a disaster, maintaining communication is important for coordinating rescue and relief efforts. IoT devices can establish ad-hoc communication networks using quantum location data to ensure robust and accurate positioning of network nodes. This ensures that first responders and emergency services remain connected even when traditional communication infrastructure is damaged.

Examples of the present disclosure may also be leveraged to optimize 5G/6G/Next-G quantum satellite-based networks. 5G/6G/Next-G Quantum satellite-based networks use quantum communication technologies, such as quantum key distribution (QKD) and entanglement-based communication, to provide highly secure and reliable communication channels. Satellites equipped with quantum communication devices (e.g., quantum sensors) can establish secure links with ground stations (e.g., ACIs) and other satellites, creating a global quantum communication network.

QKD, for instance, allows the secure generation and distribution of cryptographic keys between parties, leveraging the principles of quantum mechanics to detect any eavesdropping attempts. By integrating QKD capabilities into the satellite-based 5G/6G/Next-G network, the disclosed system can ensure that communication between nodes, including IoT devices and emergency responders, remains secure and tamper-proof. For example, during an emergency response operation, satellite-based 5G/6G/Next-G networks can use QKD to establish secure communication links between command centers and field units. This ensures that sensitive information, such as victim locations and resource deployment strategies, cannot be intercepted or altered by malicious actors.

Satellite-based 5G/6G/Next-G networks also provide global coverage, ensuring that even remote and hard-to-reach areas can maintain robust communication links. This is particularly important during large-scale disasters that might disrupt terrestrial communication infrastructure. By incorporating satellites with quantum sensors, the disclosed system can maintain communication links even if ground-based infrastructure is compromised. Satellites can serve as backup communication nodes, rerouting data through space to maintain connectivity. For example, in the event of a widespread natural disaster that damages terrestrial communication infrastructure, quantum satellite-based 5G/6G/Next-G networks can ensure continuous communication between emergency responders and command centers. Satellites can provide alternative communication paths, maintaining network functionality and supporting coordinated rescue efforts.

Quantum satellites equipped with quantum positioning systems can also provide highly accurate location data for IoT devices, vehicles, and personnel. This data enhances the overall precision and reliability of the 5G/6G/Next-G network optimization system. For example, during a search and rescue mission, quantum satellites can provide real-time location data for rescue teams and victims, ensuring that resources are deployed efficiently and accurately. The combination of quantum location data and satellite coverage ensures that even the most remote areas are effectively monitored and managed.

In another example, the quantum-classical computing module 118 of FIGS. 1A and 1B interfaces with quantum satellite-based 5G/6G/Next-G networks to process and optimize data in real-time. Quantum computing resources can handle complex calculations and predictive models, while classical systems manage data aggregation and initial processing. For instance, in an emergency scenario, the quantum-classical computing module 118 can analyze real-time data from satellites with quantum sensors and ground-based quantum sensors to predict the spread of a disaster, such as a wildfire. This information helps emergency responders make informed decisions about resource allocation and evacuation routes.

In another example, IoT quantum sensors integrated into the satellite-based 5G/6G/Next-G network can leverage secure communication channels and precise location data to enhance their functionality. This integration supports a wide range of public safety applications, from environmental monitoring to health tracking. For instance, environmental quantum sensors deployed in remote areas can transmit data securely via satellites, providing real-time updates on conditions such as air quality, radiation levels, or water contamination. This information supports proactive measures to protect public health and safety.

In another example, satellite-based 5G/6G/Next-G networks provide redundant communication paths, ensuring that data can be rerouted through space if terrestrial links fail. This redundancy enhances the fault tolerance of the overall communication system. For instance, if a terrestrial communication node is damaged during an earthquake, quantum satellites can automatically reroute data to maintain connectivity. This ensures that emergency communication remains uninterrupted, supporting effective coordination and response.

In another example, during a large-scale natural disaster, such as a hurricane, quantum satellite-based 5G/6G/Next-G networks can ensure continuous and secure communication between emergency responders and command centers. Satellites provide real-time location data from quantum sensors and secure communication channels using QKD. The disclosed system, enhanced by quantum computing, can process this real-time location data to optimize resource allocation and predict the disaster's impact. IoT devices deployed in the affected area can transmit environmental data through satellites, thereby providing important information for emergency management. Redundant communication paths via satellites ensure that connectivity is maintained even if ground-based infrastructure is compromised.

FIG. 3 depicts a high-level block diagram of a computing device or processing system specifically programmed to perform the functions described herein. As depicted in FIG. 3, the processing system 300 comprises one or more hardware processor elements 302 (e.g., at least one central processing unit (CPU), a microprocessor, or a multi-core processor), a memory 304 (e.g., random access memory (RAM) and/or read only memory (ROM)), a module 305 for optimizing the provision of emergency series in a communications network, and various input/output devices 306 (e.g., storage devices, including but not limited to, a tape drive, a floppy drive, a hard disk drive or a compact disk drive, a receiver, a transmitter, a speaker, a display, a speech synthesizer, an output port, an input port and a user input device (such as a keyboard, a keypad, a mouse, a microphone and the like)). Although only one processor element is shown, it should be noted that the computing device may employ a plurality of processor elements. Furthermore, although only one computing device is shown in the figure, if the method 200 as discussed above is implemented in a distributed or parallel manner for a particular illustrative example, i.e., the steps of the above method 200 or the entire method 200 is implemented across multiple or parallel computing devices, e.g., a processing system, then the computing device of this figure is intended to represent each of those multiple computing devices.

Furthermore, one or more hardware processors can be utilized in supporting a virtualized or shared computing environment. The virtualized computing environment may support one or more virtual machines representing computers, servers, or other computing devices. In such virtualized virtual machines, hardware components such as hardware processors and computer-readable storage devices may be virtualized or logically represented. The hardware processor 302 can also be configured or programmed to cause other devices to perform one or more operations as discussed above. In other words, the hardware processor 302 may serve the function of a central controller directing other devices to perform the one or more operations as discussed above.

It should be noted that the present disclosure can be implemented in software and/or in a combination of software and hardware, e.g., using application specific integrated circuits (ASIC), a programmable gate array (PGA) including a Field PGA, or a state machine deployed on a hardware device, a computing device or any other hardware equivalents, e.g., computer readable instructions pertaining to the method discussed above can be used to configure a hardware processor to perform the steps, functions and/or operations of the above disclosed method 200. In one example, instructions and data for the present module or process 305 for optimizing the provision of emergency series in a communications network (e.g., a software program comprising computer-executable instructions) can be loaded into memory 304 and executed by hardware processor element 302 to implement the steps, functions, or operations as discussed above in connection with the illustrative method 200. Furthermore, when a hardware processor executes instructions to perform “operations,” this could include the hardware processor performing the operations directly and/or facilitating, directing, or cooperating with another hardware device or component (e.g., a co-processor and the like) to perform the operations.

The processor executing the computer readable or software instructions relating to the above described method can be perceived as a programmed processor or a specialized processor. As such, the present module 305 for optimizing the provision of emergency series in a communications network (including associated data structures) of the present disclosure can be stored on a tangible or physical (broadly non-transitory) computer-readable storage device or medium, e.g., volatile memory, non-volatile memory, ROM memory, RAM memory, magnetic or optical drive, device or diskette, and the like. Furthermore, a “tangible” computer-readable storage device or medium comprises a physical device, a hardware device, or a device that is discernible by the touch. More specifically, the computer-readable storage device may comprise any physical devices that provide the ability to store information such as data and/or instructions to be accessed by a processor or a computing device such as a computer or an application server.

While various examples have been described above, it should be understood that they have been presented by way of illustration only, and not a limitation. Thus, the breadth and scope of any aspect of the present disclosure should not be limited by any of the above-described examples, but should be defined only in accordance with the following claims and their equivalents.

What is claimed is:

1. A method comprising:
collecting, by a processing system including at least one processor from a quantum sensor in a communications network, position data for a public safety device;
tracking, by the processing system, a movement pattern and a usage pattern of the public safety device by providing the position data as input to a quantum machine learning model that generates the movement pattern and the usage pattern as an output;
determining, by the processing system based on an analysis of the movement pattern and the usage pattern, that an emergency service should be contacted; and
placing, by the processing system, a call to the emergency service.

2. The method of claim 1, wherein the public safety device comprises one of: an automated external defibrillator, a fire extinguisher, an epinephrine auto-injector, an inhaler, a fire alarm, a carbon monoxide detector, a mobile communications device, a medical monitoring device, an internet of things-enabled medical device, or an environmental sensor.

3. The method of claim 1, wherein the quantum sensor transmits the position data to the processing system via an agent-computer interface.

4. The method of claim 1, wherein the position data comprises a stream of physical positions of the public safety device, wherein each physical position in the stream of physical positions is timestamped.

5. The method of claim 4, wherein each physical position is defined by a latitude coordinate, a longitude coordinate, and an altitude coordinate.

6. The method of claim 1, wherein the quantum machine learning model is executed by at least one of: a quantum computing module or a quantum-classical computing module in the communications network.

7. The method of claim 1, wherein the usage pattern comprises an indication as to whether the public safety device has been used.

8. The method of claim 7, wherein the quantum machine learning model determines whether the public safety device has been used based on monitoring of a power level of the public safety device.

9. The method of claim 1, wherein the usage pattern comprises readings from the public safety device.

10. The method of claim 9, wherein the readings comprise carbon monoxide levels, blood glucose measurements, or smoke levels.

11. The method of claim 1, wherein the analysis indicates that the public safety device has been moved from a default position and that the public safety device has been used since being moved from the default position.

12. The method of claim 1, wherein the placing comprises engaging an agent running on a computer to place at least one of: a voice call, a video call, a text message using a communications device.

13. The method of claim 12, wherein the engaging further comprises utilizing at least one of: a large language model or a speech generation technology to generate a synthesized audio output to provide to the emergency service via the call.

14. The method of claim 13, wherein the synthesized audio output includes at least one of: a type of the public safety device, a current position of the public safety device, a time at which the public safety device was used, a position of the public safety device at the time at which the public safety device was used, or readings measured by the public safety device.

15. The method of claim 1, wherein the placing further comprises plotting the position data of the public safety device on a three-dimensional map to provide to the emergency service.

16. The method of claim 15, wherein the three-dimensional map indicates at least one of: a current position of the public safety device or a position of the public safety device at a time at which the public safety device was inferred to have been used based on the usage pattern.

17. The method of claim 16, wherein the three-dimensional map indicates the at least one of: the current position of the public safety device or the position of the public safety device at a time at which the public safety device was inferred to have been used as a set of latitude, longitude, and altitude coordinates.

18. The method of claim 17, wherein the three-dimensional map shows the at least one of: the current position of the public safety device or the position of the public safety device at a time at which the public safety device was inferred to have been used within a multistory building.

19. A non-transitory computer-readable medium storing instructions which, when executed by a processing system including at least one processor, cause the processing system to perform operations, the operations comprising:
collecting, from a quantum sensor in a communications network, position data for a public safety device;
tracking a movement pattern and a usage pattern of the public safety device by providing the position data as input to a quantum machine learning model that generates the movement pattern and the usage pattern as an output;
determining, based on an analysis of the movement pattern and the usage pattern, that an emergency service should be contacted; and
placing a call to the emergency service.

20. A device comprising:
a processing system including at least one processor; and
a non-transitory computer-readable medium storing instructions which, when executed by the processing system, cause the processing system to perform operations, the operations comprising:
collecting, from a quantum sensor in a communications network, position data for a public safety device;
tracking a movement pattern and a usage pattern of the public safety device by providing the position data as input to a quantum machine learning model that generates the movement pattern and the usage pattern as an output;
determining, based on an analysis of the movement pattern and the usage pattern, that an emergency service should be contacted; and
placing a call to the emergency service.
